(19) United States
(12) Patent Application Publication
(10) Pub. No.: US 2010/0144728 A1
(43) Pub. Date: Jun. 10, 2010

(54) Oxazolidinone For The Treatment And Prophylaxis Of Pulmonary Hypertension

(75) Inventors: Georges Von Degenfeld, Leverkusen (DE); Martina Klein, Heiligenhaus (DE); Elisabeth Perzborn, Wuppertal (DE); Joachim Hütter, Berlin (DE); Gerrit Weimann, Koln (DE)

Correspondence Address: CONNOLLY BOVE LODGE & HUTZ, LLP, P O BOX 2207, WILMINGTON, DE, 19899, US

(73) Assignee: Bayer Schering Pharma AG, Berlin (DE)

(21) Appl. No.: 12/596,557

(22) PCT Filed: Apr. 10, 2008
(86) PCT No.: PCT/EP08/02829

§ 371 Date: Oct. 19, 2009

(30) Foreign Application Priority Data
Apr. 20, 2007 (DE) 102007018662.4

(51) Int. Cl. A61K 31/5377; A61P 9/12

(52) U.S. Cl. 514/230.8

(57) ABSTRACT

The present invention relates to the use of factor Xa inhibitors, especially of oxazolidinones of the formula (I), for the treatment and/or prophylaxis of pulmonary hypertension, and to the use thereof for the manufacture of medicaments for the treatment and/or prophylaxis of pulmonary hypertension.

The present invention relates to the use of selective factor Xa inhibitors, especially of oxazolidinones of the formula (I), for the treatment and/or prophylaxis of pulmonary hypertension, and to the use thereof for the manufacture of medicaments for the treatment and/or prophylaxis of pulmonary hypertension.

Oxazolidinones of the formula (I) are disclosed in WO 01/047919 and act in particular as selective inhibitors of coagulation factor Xa and as anticoagulants.

Oxazolidinones of the formula (I) are selective factor Xa inhibitors and specifically inhibit only FXa. It has been possible to demonstrate an antithrombotic effect of factor Xa inhibitors in numerous animal models (cf. U. Sinha, P. Ku, J. Malinowski, B. Yan Zhu, R M. Scarborough, C K. Marlowe, P W. Wong, P. Hua Lin, S J. Hollenbach, Antithrombotic and hemostatic capacity of factor Xa versus thrombin inhibitors in models of venous and arteriovenous thrombosis, European Journal of Pharmacology 2000, 395, 51-59; A. Betz, Recent advances in Factor Xa inhibitors, Expert Opin. Ther. Patents 2001, 11, 1007; K. Tsong Tan, A. Makin, G. Y H Lip, Factor X inhibitors, Exp. Opin. Investig. Drugs 2003, 12, 799; J. Ruef, H A. Katus, New antithrombotic drugs on the horizon, Expert Opin. Investig. Drugs 2003, 12, 781; M M. Samama, Synthetic direct and indirect factor Xa inhibitors, Thrombosis Research 2002, 106, V267; M L. Quan, J M. Smallheer, The race to an orally active Factor Xa inhibitor, Recent advances, J. Current Opinion in Drug Discovery & Development 2004, 7, 460-469) and in clinical studies on patients (The Study, Blood 2000, 96, 490a; The Penthifra Study, Blood 2000, 96, 490a; The Pentamaks Study, Blood 2000, 96, 490a-491a; The Pentathlon 2000 Study, Blood 2000, 96, 491a). Factor Xa inhibitors can therefore be employed preferably in medicaments for the prophylaxis and/or treatment of thromboembolic disorders. Selective FXa inhibitors show a broad therapeutic window. It has been possible to show in numerous animal experimental investigations that FXa inhibitors show an antithrombotic effect in models of thrombosis without a, or with only a slight, prolonging effect on bleeding times (compare R J Leadly, Coagulationfactor Xa biological background and rationale, Curr Top Med Chem 2001; 1, 151-159). Individual dosage for anticoagulation with selective FXa inhibitors is therefore unnecessary.

Pulmonary hypertension (Clinical Classification of Pulmonary Hypertension, Venice 2003) is a progressive lung disorder which may have various causes and, untreated, results in death. It is associated with an overload on the right heart with right heart failure progressing to pump failure, which may result in death. By definition, in chronic pulmonary hypertension the mean pulmonary artery pressure (mPAP) is >25 mmHg at rest and >30 mmHg during exercise (normal value <20 mmHg). The pathophysiology of pulmonary hypertension in many cases also includes thrombosis of the pulmonary vessels. Pulmonary arterial hypertension may be associated with an increase in the intima and media (inner and middle layer of the vessel wall) and with thrombosis, followed by a slow transformation of muscle into connective tissue. This increasing obliteration of the pulmonary circulation results in a progressive stress on the right heart, leading to a reduced output by the right heart and finally terminating in right heart failure.

So-called primary pulmonary hypertension (PAH), which occurs without identifiable cause, is an extremely rare disorder with a prevalence of 1-2 per million (G. E. D'Alonzo et al., Ann. Intern. Med. 1991, 115, 343-349). The average age of the patients has been estimated to be 36 years, and only 10% of the patients were over 60 years of age. Distinctly more women than men are affected. The secondary forms of pulmonary hypertension show, consistent with the diversity of the causes underlying them, different courses, but in every case it is a severe disorder with high mortality.

Anticoagulation with a vitamin K antagonist (warfarin) is recommended (American College of Chest Physicians) for primary pulmonary hypertension. A chronic treatment with warfarin is also carried out in many cases for other, secondary forms of pulmonary hypertension. Although a benefit of this therapy has not to date been investigated in appropriate (prospective randomized and double-blind) clinical studies, smaller observation studies indicate distinct survival advantages for patients taking warfarin compared with patients receiving no anticoagulants. Heparins have been investigated in the animal experimental model of primary pulmonary arterial hypertension through injection of monocrotaline (MCT) in rats, a standard model for this disorder, but show no positive effect.

Despite all the advances in the therapy of pulmonary hypertension, there is as yet no prospect of curing this serious disorder. Specific therapies available on the market for pulmonary hypertension (e.g. prostacyclin analogues, endothelin receptor antagonists, phosphodiesterase inhibitors) are, however, able to improve the quality of life, the exercise tolerance and the prognosis of the patients. However, the usability of these medicaments is restricted by the in some cases serious side effects and/or complicated administration forms. The period over which the patients' clinical situation can be improved or stabilized with a specific therapy is limited. Eventually, the therapy escalates and thus a combination therapy is applied, where a plurality of medicaments must be given concurrently. Novel combination therapies are one of the most promising future therapeutic options for the treatment of pulmonary arterial hypertension (Ghofrani et al., Herz 2005, 30, 296-302). It is increasingly important in the development of novel therapies for them to be combinable with known ones and not generate any problems associated with metabolism, e.g. inhibit P450 CYP enzymes to only a very small extent or not at all (compare medicament interactions associated with combination therapy with bosentan and warfarin).

WO 2006/045756 mentions the combination of dipyridamole with rivaroxaban for the treatment of pulmonary hypertension, but without mentioning any findings. However, dipyridamole has a large number of side effects such as, for example, hypotension, cardiac arrest, cardiac dysrhythmias, allergic reactions/deterioration in bronchial asthma (to tartrazine in the case of hypersensitivity), bronchial asthma, liver enzyme elevation and hepatic failure. In addition, dipyridamole shows interactions with other medicaments such as, for example, platelet aggregation inhibitors (e.g. aspirin) or anticoagulants (e.g. warfarin).

It is therefore desirable to find compounds which can be employed for the therapy of pulmonary hypertension such that the therapy leads to a normalization or marked improvement in relevant parameters such as, for example, right ventricular pressure, pulmonary artery pressure, exercise tolerance and mixed venous oxygen saturation, brings about a marked simplification in the treatment, improves the tolerability and reduces the risk through avoidance of side effects, can be used for a larger number of patients and reduces interactions with other medicaments.

It has now surprisingly been found that selective factor Xa inhibitors, especially oxazolidinones of the formula (I), are suitable for the treatment and prevention of pulmonary hypertension, especially pulmonary arterial hypertension.

The present invention relates to the use of selective factor Xa inhibitors for the manufacture of medicaments for the treatment and/or prophylaxis of pulmonary hypertension, especially pulmonary arterial hypertension.

The present invention relates in particular to the use of compounds of the formula (I)

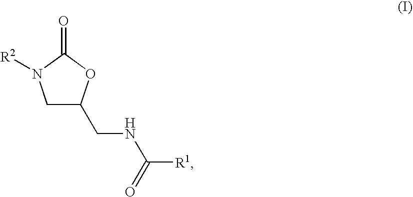

in which
R¹ is 2-thiophene which is substituted in position 5 by a radical selected from the group of chlorine, bromine, methyl and trifluoromethyl,
R² is D-A-,
   where
   the radical “A” is phenylene,
      where
      the group “A” may be substituted where appropriate once or twice in the meta position relative to the linkage to the oxazolidinone by a radical selected from the group of fluorine, chlorine, nitro, amino, trifluoromethyl, methyl and cyano,
   and
   the radical “D” is a saturated 5- or 6-membered heterocycle which is linked via a nitrogen atom to “A” and has in the direct vicinity of the linking nitrogen atom a carbonyl group, and in which one ring carbon member may be replaced by a heteroatom from the series S, N and O,
      and the salts, solvates and solvates of the salts thereof for the manufacture of medicaments for the treatment and/or prophylaxis of pulmonary hypertension.

Very particular preference is given in this connection to the use of the compound 5-chloro-N-({(5S)-2-oxo-3-[4-(3-oxomorpholin-4-yl)phenyl]-1,3-oxazolidin-5-yl}methyl)thiophene-2-carboxamide (Example 1) having the following formula

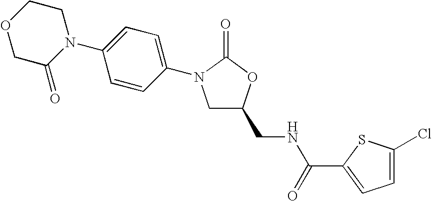

and the salts, solvates and solvates of the salts thereof for the manufacture of medicaments for the treatment and/or prophylaxis of pulmonary hypertension.

Oxazolidinones were originally described essentially only as antibiotics, and in a few cases as MAO inhibitors and fibrinogen antagonists (Review: Riedl, B., Endermann, R., Exp. Opin. Ther. Patents 1999, 9 (5), 625), a small 5-[acylaminomethyl] group (preferably 5-[acetylaminomethyl]) apparently being essential for the antibacterial effect.

Substituted aryl- and heteroarylphenyloxazolidinones in which a mono- or polysubstituted phenyl radical may be bonded to the N atom of the oxazolidinone ring and which may have an unsubstituted N-methyl-2-thiophenecarboxamide residue in position 5 of the oxazolidinone ring, and the use thereof as substances with antibacterial activity are disclosed in U.S. Pat. No. 5,929,248, U.S. Pat. No. 5,801,246, U.S. Pat. No. 5,756,732, U.S. Pat. No. 5,654,435, U.S. Pat. No. 5,654,428 and U.S. Pat. No. 5,565,571.

In addition, benzamidine-containing oxazolidinones are known as synthetic intermediates in the synthesis of factor Xa inhibitors and fibrinogen antagonists (WO 99/31092, EP 0 623 615).

Compounds which can be used according to the invention, also referred to hereinafter as compounds of the invention, are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, in so far as the compounds encompassed by formula (I) and mentioned hereinafter are not already salts, solvates and solvates of the salts.

The compounds of the invention may, depending on their structure, exist in stereoisomeric forms (enantiomers, diastereomers). The invention therefore includes the use of the enantiomers or diastereomers and respective mixtures thereof.

Where the compounds of the invention can occur in tautomeric forms, the present invention encompasses the use of all tautomeric forms.

Salts preferred in the context of the present invention are physiologically acceptable salts of the compounds of the invention. Also included are salts which are themselves unsuitable for pharmaceutical applications but can be used for example for isolating or purifying the compounds of the invention.

Physiologically acceptable salts of the compounds of the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as by way of example and preferably alkali metal salts (e.g. sodium and potassium salts), alkaline earth metal salts (e.g. calcium and magnesium salts) and ammonium salts derived from ammonia or organic amines having 1 to 16 C atoms, such as by way of example and preferably ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refer in the context of the invention to those forms of the compounds of the invention which form a complex in the solid or liquid state through coordination with solvent molecules. Hydrates are a specific form of solvates in which the coordination takes place with water. Solvates preferred in the context of the present invention are hydrates.

The present invention additionally also includes the use of prodrugs of the compounds of the invention. The term “prodrugs” includes compounds which themselves may be biologically active or inactive but are converted (for example by metabolism or hydrolysis) during their residence time in the body into compounds of the invention.

In the context of the present invention, the substituents have the following meaning unless specified otherwise:

A saturated 5- or 6-membered heterocycle which is linked via a nitrogen atom and has in the direct vicinity of the linking nitrogen atom a carbonyl group, and in which a ring carbon member may be replaced by a heteroatom from the series S, N and O, is for example 2-oxopyrrolidin-1-yl, 2-oxo-piperidin-1-yl, 2-oxopiperazin-1-yl, 2-oxomorpholin-1-yl, 3-oxothiomorpholin-4-yl, 2-oxo-1,3-oxazolidin-1-yl, 2-oxo-1,3-oxazinan-1-yl, 2-oxoimidazolidin-1-yl and 2-oxotetrahydropyrimidin-1-yl.

### EXPLANATIONS OF THE FIGURES

FIG. 1: Maximum right-ventricular pressure

FIG. 2: Maximum right-ventricular pressure

FIG. 3: Right-ventricular hypertrophy

FIG. 4: Right-ventricular hypertrophy

FIG. 5: right-ventricular end-diastolic pressure (RVEDP)

FIG. 6: right-ventricular end-diastolic pressure (RVEDP)

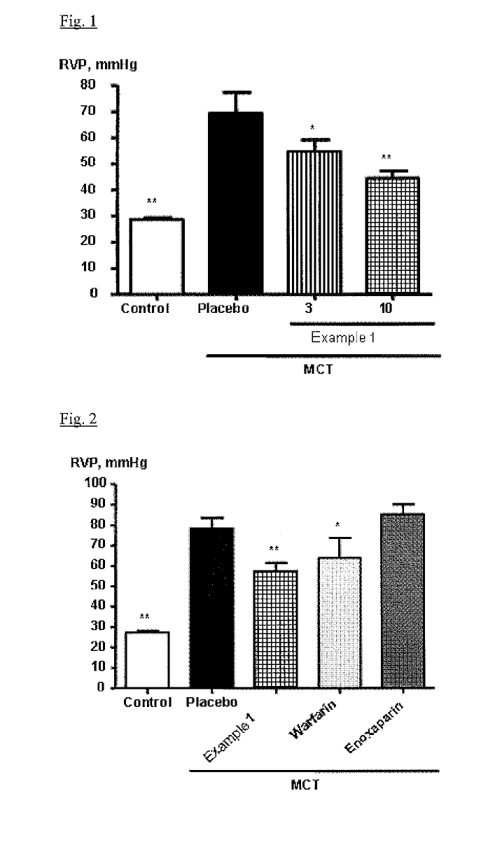

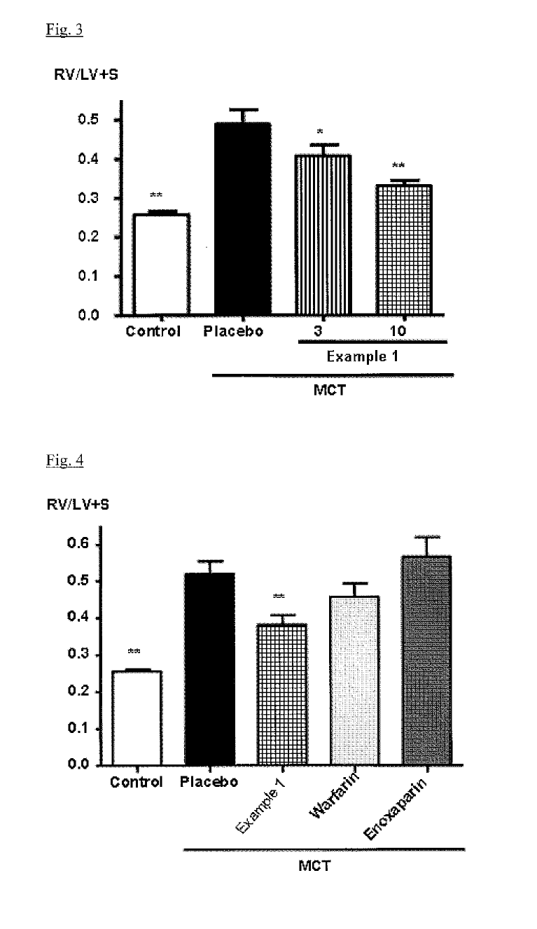

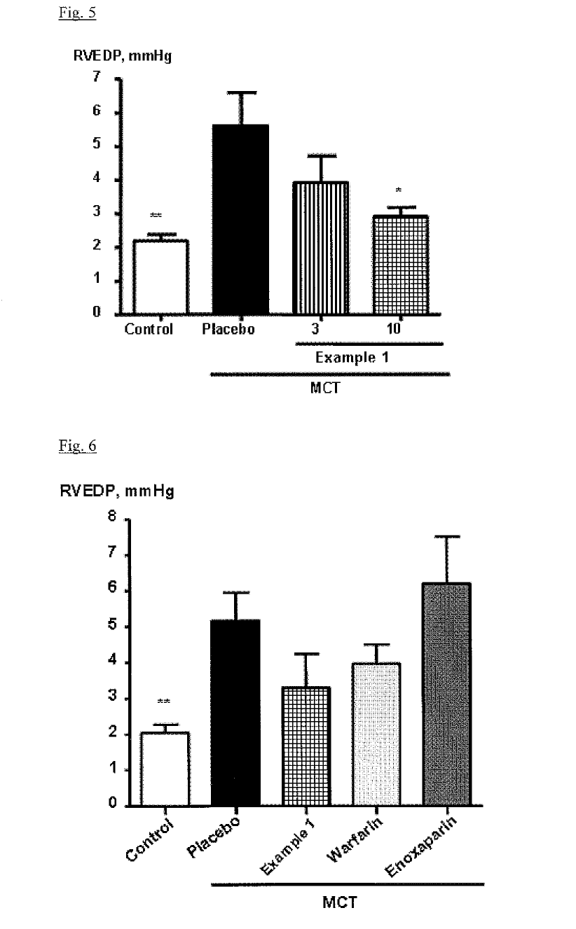

The compounds of the formula (I) can be prepared by either

[A] compounds of the general formula

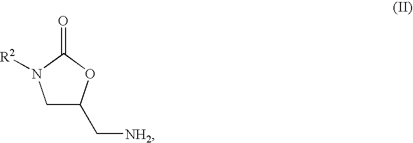

in which
R² has the meaning indicated above,
being reacted with carboxylic acids of the general formula

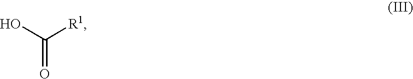

in which
R¹ has the meaning indicated above,
or else with the corresponding carbonyl halides, preferably carbonyl chlorides, or else with the corresponding symmetrical or mixed carboxylic anhydrides of the carboxylic acids of the general formula (III) defined above
in inert solvents, where appropriate in the presence of an activating or coupling reagent and/or of a base,
   or
   [B] compounds of the general formula

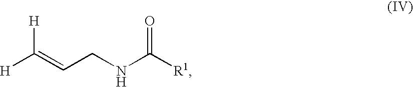

in which
R¹ has the meaning indicated above,
being converted with a suitable selective oxidizing agent in an inert solvent into the corresponding epoxide of the general formula

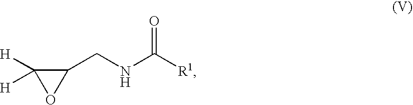

in which
R¹ has the meaning indicated above,
and the of the general formula

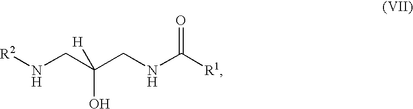

in which
R¹ and R² have the meaning indicated above,
firstly being prepared by reaction, in an inert solvent where appropriate in the presence of a catalyst, with an amine of the general formula

R²—NH₂ (VI),
in which
R² has the meaning indicated above, and
subsequently being cyclized in an inert solvent in the presence of phosgene or phosgene equivalents such as, for example, carbonyldiimidazole (CDI) to give the compounds of the general formula (I).

Suitable solvents for the processes described above are in this ease organic solvents which are inert under the reaction conditions. These include halohydrocarbons such as dichloromethane, trichloromethane, tetrachloromethane, 1,2-dichloroethane, trichloroethane, tetrachloroethane, 1,2-dichloroethylene or trichloroethylene, ethers such as diethyl ether, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane or cyclohexane, dimethylformamide, dimethyl sulphoxide, acetonitrile, pyridine, hexa-methylphosphoric triamide or water. It is likewise possible to employ solvent mixtures of the aforementioned solvents.

Suitable activating or coupling reagents for the processes described above are in this case the reagents normally used for this purpose, for example N′-(3-dimethylaminopropyl)-N-ethylcarbodiimide.HCl, N,N′-dicyclohexylcarbodiimide, 1-hydroxy-1H-benzotriazole.H₂O and the like.

Suitable bases are the usual inorganic or organic bases. These preferably include alkali metal hydroxides such as, for example, sodium or potassium hydroxide or alkali metal carbonates such as sodium or potassium carbonate or sodium or potassium methanolate or sodium or potassium ethanolate or potassium tert-butoxide or amides such as sodium amide, lithium bis-(trimethylsilyl)amide or lithium diisopropylamide or amines such as triethylamine, diisopropylethylamine, diisopropylamine, 4-N,N-dimethylaminopyridine or pyridine.

The base can in this case be employed in an amount of from 1 to 5 mol, preferably from 1 to 2 mol, based on 1 mol of the compounds of the general formula (II).

The reactions generally take place in a temperature range from −78° C. to the reflux temperature, preferably in the range from 0° C. to the reflux temperature.

The reactions can be carried out under atmospheric, elevated or reduced pressure (e.g. in the range from 0.5 to 5 bar). They are generally carried out under atmospheric pressure.

Suitable selective oxidizing agents both for preparing the epoxides and for the oxidation, which is carried out where appropriate, to the sulphone, sulphoxide or N-oxide are for example m-chloroperbenzoic acid (MCPBA), sodium metaperiodate, N-methylmorpholine N-oxide (NMO), monoperoxyphthalic acid or osmium tetroxide.

The epoxides are prepared by employing the preparation conditions customary for this purpose.

Concerning the detailed process conditions for the oxidation, which is carried out where appropriate, to the sulphone, sulphoxide or N-oxide, reference may be made to the following literature: M. R. Barbachyn et al., J. Med. Chem. 1996, 39, 680 and WO 97/10223.

The compounds of the formulae (II), (III), (IV) and (VI) are known per se to the person skilled in the art or can be prepared by conventional methods. For oxazolidinones, especially the required 5-(aminomethyl)-2-oxooxazolidines, cf. WO 98/01446; WO 93/23384; WO 97/03072; J. A. Tucker et al., J. Med. Chem. 1998, 41, 3727; S. J. Brickner et al., J. Med. Chem. 1996, 39, 673; W. A. Gregory et al., J. Med. Chem. 1989, 32, 1673.

The processes for synthesizing oxazolidinones of the formula (I) are described in detail in WO 01/047919.

The term “pulmonary hypertension” includes particular forms of pulmonary hypertension as specified for example by the World Health Organisation (WHO) (Clinical Classification of Pulmonary Hypertension, Venice 2003). Examples which may be mentioned are pulmonary arterial hypertension, pulmonary hypertension associated with left heart disorders, pulmonary hypertension associated with lung disease and/or hypoxia and pulmonary hypertension due to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary arterial hypertension (APAH) which is associated with collagenoses, congenital systemic-pulmonary shunts, portal hypertension, HIV infections, intake of particular drugs and medicaments, with other disorders (thyroid disorders, glycogen storage diseases, Gaucher's disease, hereditary telangiectasia, haemoglobinopathies, myeloproliferative disorders, splenectomy), with disorders with significant venous/capillary involvement such as pulmonary venoocclusive disease and pulmonary capillary haemangiomatosis, and persistent pulmonary hypertension of newborns.

Pulmonary hypertension associated with left heart disorders includes disorders of the left atrium or ventricle and mitral or aortic valve defects.

Pulmonary hypertension associated with lung disease and/or hypoxia includes chronic obstructive pulmonary disorders, interstitial lung disease, sleep apnoea syndrome, alveolar hypoventilation, chronic altitude sickness and constitutional abnormalities.

Pulmonary hypertension due to chronic thromboembolisms (CTEPH) includes thromboembolic obstruction of proximal pulmonary arteries, thromboembolic obstruction of distal pulmonary arteries and non-thrombotic pulmonary embolisms (tumour, parasites, foreign bodies).

The present invention further relates to the use of selective factor Xa inhibitors for the manufacture of medicaments for the treatment and/or prophylaxis of pulmonary hypertension associated with sarcoidosis, histiocytosis X and lymphangiomatosis.

The present invention further relates to medicaments comprising a compound according to the invention and one or more further active ingredients, especially for the treatment and/or prophylaxis of the aforementioned disorders. Examples of suitable combination active ingredients which may preferably be mentioned are:
lipid-lowering agents, especially HMG-CoA (3-hydroxy-3-methylglutarylcoenzyme A) reductase inhibitors;
coronary therapeutics/vasodilators, especially ACE (angiotensin converting enzyme) inhibitors, AII (angiotensin II) receptor antagonists; β-adrenoceptor antagonists; alpha-1 adrenoceptor antagonists; diuretics; calcium channel blockers; substances which bring about an increase in cyclic guanosine monophosphate (cGMP), such as, for example, stimulators of soluble guanylate cyclase;
plasminogen activators (thrombolytics/fibrinolytics) and compounds which increase thrombolysis/fibrinolysis, such as inhibitors of plasminogen activator inhibitor (PAI inhibitors) or inhibitors of the thrombin-activated fibrinolysis inhibitor (TAFI inhibitors);
substances having anticoagulant activity (anticoagulants);
platelet aggregation-inhibiting substances (platelet aggregation inhibitors);
fibrinogen receptor antagonists (glycoprotein antagonists);
antiarrhythmics;
kinase inhibitors;
stimulators and activators of soluble guanylate cyclase;
prostacyclin analogues;
endothelin receptor antagonists;
and phosphodiesterase inhibitors.

The present invention further relates to a method for the treatment and/or prophylaxis of pulmonary hypertension in humans and animals by administering an effective amount of at least one selective factor Xa inhibitor or of a medicament comprising at least one selective factor Xa inhibitor in combination with an inert, non-toxic, pharmaceutically suitable excipient.

The present invention further relates to a method for the treatment and/or prophylaxis of pulmonary hypertension in humans and animals through administration of an effective amount of at least one compound of the invention, or of a medicament comprising at least one compound of the invention, in combination with an inert, non-toxic, pharmaceutically suitable excipient.

The medicaments to be manufactured in accordance with the use according to the invention or to be used according to the invention comprise at least one compound of the invention, normally together with one or more inert, non-toxic, pharmaceutically suitable excipients.

The compounds according to the invention can act systemically and/or locally. For this purpose, they can be administered in a suitable way such as, for example, by the oral, parenteral, pulmonary, nasal, sublingual, lingual, buccal, rectal, dermal, transdermal, conjunctival or otic route or as implant or stent.

The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays, tablets, films/wafers or capsules to be administered by the lingual, sublingual or buccal route, suppositories, preparations for the eyes or ears, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Oral or parenteral administration is preferred, especially oral administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavours and/or odours.

It has generally proved advantageous to administer on parenteral administration amounts of about 0.001 to 1 mg/kg, preferably about 0.01 to 0.5 mg/kg, of body weight to achieve effective results. On oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are, unless indicated otherwise, in each case based on volume.

### EXAMPLES

### A. Preparation Examples

### Starting Compounds

The syntheses of the starting compounds are described in detail in WO 01/047919.

### Exemplary Embodiments

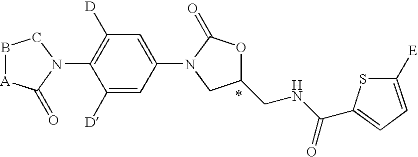

| Example | A—B—C | D | D′ | E |
|---|---|---|---|---|
| 1 | CH₂OCH₂CH₂ | H | H | Cl |
| 2 | CH₂CH₂CH₂ | H | H | Cl |
| 3 | CH₂CH₂CH₂ | H | H | CH₃ |
| 4 | CH₂CH₂CH₂ | H | H | Br |
| 5 | CH₂OCH₂CH₂ | H | H | CH₃ |
| 6 | CH₂OCH₂CH₂ | H | H | Br |
| 7 | OCH₂CH₂ | H | H | Cl |
| 8 | CH₂CH₂CH₂ | H | H | Br |
| 9 | CH₂CH₂CH₂ | H | H | CH₃ |
| 10 | OCH₂CH₂CH₂ | H | H | Cl |
| 11 | CH₂CH₂CH₂ | F | H | Cl |
| 12 | CH₂OCH₂CH₂ | H | H | Cl |
| 13 | CH₂CH₂CH₂ | CF₃ | H | Cl |
| 14 | CH₂OCH₂CH₂ | Cl | H | Cl |
| 15 | CH₂OCH₂CH₂ | CF₃ | H | Cl |
| 16 | CH₂OCH₂CH₂ | CH₃ | H | Cl |
| 17 | CH₂OCH₂CH₂ | CN | H | Cl |
| 18 | CH₂CH₂CH₂ | Cl | H | Cl |
| 19 | CH₂OCH₂CH₂ | CH₃ | CH₃ | Cl |
| 20 | CH₂OCH₂CH₂ | NH₂ | H | Cl |
| 21 | CH₂OCH₂CH₂ | F | H | Br |
| 22 | CH₂CH₂CH₂ | F | H | Br |
| 23 | CH₂CH₂CH₂CH₂ | H | H | Br |
| 24 | CH₂CH₂CH₂ | F | H | Cl |
| 25 | CH₂OCH₂CH₂ | F | H | Cl |
| 26 | CH₂CH₂CH₂CH₂ | H | H | Cl |

The syntheses of the exemplary embodiments are described in detail in WO 01/047919.

### B. Assessment of the Physiological Activity

The compounds of the formula (I) act in particular as selective inhibitors of coagulation factor Xa and do not inhibit, or else inhibit only at distinctly higher concentrations, other serine proteases such as plasmin or trypsin.

Inhibitors of coagulation factor Xa are referred to as “selective” when their IC₅₀ values for factor Xa inhibition are at least 100-fold smaller than the IC₅₀ values for the inhibition of other serine proteases, in particular plasmin and trypsin, reference being made concerning the test methods for the selectivity to the test methods of Examples A.a.1) and A.a.2) described below.

Advantageous pharmacological properties of the compounds which can be used according to the invention can be ascertained by the following methods.

### a) Test Description (In Vitro)

### a.1) Measurement of Factor Xa Inhibition

The enzymatic activity of human factor Xa (FXa) was measured via the conversion of an FXa-specific chromogenic substrate. In this case, factor Xa eliminates p-nitroaniline from the chromogenic substrate. The determinations were carried out in microtitre plates as follows.

The test substances were dissolved in various concentrations in DMSO and incubated with human FXa (0.5 nmol/l dissolved in 50 mmol/l tris buffer [C,C,C-tris(hydroxymethyl)aminomethane], 150 mmol/l NaCl, 0.1% BSA (bovine serum albumin), pH=8.3) at 25° C. for 10 minutes. Pure DMSO serves as control. The chromogenic substrate (150 μmol/l Pefachrome® FXa from Pentapharm) was then added. After incubation at 25° C. for 20 minutes, the extinction at 405 nm was determined. The extinctions of the test mixtures with test substance were compared with the control mixtures without test substance, and the IC₅₀ values were calculated therefrom.

### a.2) Selectivity Determination

Selective FXa inhibition was demonstrated by investigating the inhibition by the test substances of other human serine proteases such as trypsin, plasmin. The enzymatic activity of trypsin (500 mU/ml) and plasmin (3.2 nmol/l) was determined by dissolving these enzymes in tris buffer (100 mmol/l, 20 mmol/l CaCl₂, pH=8.0) and incubating with test substance or solvent for 10 minutes. The enzymatic reaction was then started by adding the appropriate specific chromogenic substrates (Chromozym Trypsin® and Chromozym Plasmin®; from Roche Diagnostics), and the extinction was determined at 405 nm after 20 minutes. All determinations were carried out at 37° C. The extinctions of the test mixtures with test substance were compared with the control samples without test substance, and the IC₅₀ values were calculated therefrom.

### a.3) Determination of the Anticoagulant Effect

The anticoagulant effect of the test substances was determined in vitro in human and rabbit plasma. For this purpose, blood was collected using a 0.11 molar sodium citrate solution as recipient in the sodium citrate/blood mixing ratio of 1/9. The blood was thoroughly mixed immediately after collection and centrifuged at about 2500 g for 10 minutes. The supernatant was removed by pipette. The prothrombin time (PT, synonym: Quick's test) was determined in the presence of varying concentrations of test substance or the appropriate solvent using a commercially available test kit (Neoplastin® from Boehringer Mannheim or Heinoliance® RecombiPlastin from Instrumentation Laboratory). The test compounds were incubated with the plasma at 37° C. for 3 minutes. Coagulation was then induced by adding thromboplastin, and the time of onset of coagulation was determined. The concentration of test substance which brings about a doubling of the prothrombin time was found.

### b) Determination of the Antithrombotic Effect (In Vivo)

### b) Arteriovenous Shunt Model (Rat)

Fasting male rats (strain: HSD CPB:WU) weighing 200-250 g were anaesthetized with a Rompun/Ketavet solution (12 mg/kg/50 mg/kg). Thrombus formation was induced in an arteriovenous shunt by a method based on that described by Christopher N. Berry et al., Br. J. Pharmacol. (1994), 113, 1209-1214. For this purpose, the left jugular vein and the right carotid artery were exposed. The extracorporeal shunt was formed by tying in each case a 10 cm-long polyethylene tube (PE 60) into the two exposed vessels. The shunt was closed in the middle by means of a 3 cm-long polyethylene tube (PE 160) which contained a roughened nylon thread forming a loop to produce a thrombogenic surface. The extracorporeal circulation was maintained for 15 minutes. The shunt was then removed and the nylon thread with the thrombus was immediately weighed. The blank weight of the nylon thread had been found before the start of the experiment. The test substances were administered either intravenously through the tail vein or orally by gavage to conscious animals before setting up the extracorporeal circulation.

### c) Determination of the Effect on Pulmonary Hypertension (In Vivo)

The monocrotaline-induced pulmonary hypertension in rats is a widely used animal model of pulmonary arterial hypertension. The pyrrolizidine alkaloid monocrotaline is metabolized after subcutaneous injection to toxic monocrotaline pyrrole in the liver and leads within a few days to endothelial damage and coagulation activation and thrombosis formation in the pulmonary circulation, followed by remodelling of the small pulmonary arteries (media hypertrophy, de-novo muscularization). A single subcutaneous injection is sufficient to induce pronounced pulmonary hypertension in rats within 4 weeks.

Male Sprague-Dawley rats are used for the model. On day 0, the animals receive a subcutaneous injection of monocrotaline 60 mg/kg. The treatment of the animals starts before the monocrotaline injection and extends over a period of at least 28 days. At the end of the study, the animals undergo haemodynamic investigations, and the arterial and central venous oxygen saturation is measured. The rats are initially anaesthetized with pentobarbital 60 mg/kg for the haemodynamic measurement. The animals are then tracheotomized and artificially ventilated (frequency: 60 breaths/min; inspiration to expiration ratio: 50:50; positive end-expiratory pressure: 1 cm H₂O; tidal volume: 10 ml/kg body weight; FIO₂: 0.5). The anaesthesia is maintained by isoflurane inhalation anaesthesia. The systemic blood pressure is measured in the left carotid artery by means of a Millar microtip catheter. A polyethylene catheter is advanced through the right jugular vein into the right ventricle for determining the right ventricular pressure. The cardiac output is measured by thermodilution. Following the haemodynamics, the heart is removed and the ratio of right to left ventricle including septum is determined.

There was found to be a dose-dependent improvement in the haemodynamics in the right heart and in the pulmonary circulation, and in the right heart hypertrophy: maximum pulmonary arterial pressure (RVPmax), right ventricular systolic (maximum) pressure, right ventricular end-diastolic pressure (RVEDP), right ventricular maximum rate of pressure rise (dp/dtmax), the weight of the right heart relative to that of the left heart including septum (RV/(LV+S)) and the global cardiac pumping performance (cardiac output (CO)). In contrast thereto, the antithrombotic enoxaparin, a low molecular weight heparin, shows no effect, and warfarin shows a significantly smaller effect which is, however, achieved at the expense of increased bleeding complications. There were no bleedings in the animals treated with Example 1. By contrast, in each case half of the animals treated with enoxaparin or warfarin developed bleeding complications with either a fatal outcome or with the need to euthanize the animals. In summary, Example 1 shows both better activity and fewer side effects compared with enoxaparin and warfarin.

TABLE 1

| | RVPmax | RVEDP | dP/dtmax | RV/ | CO |
|---|---|---|---|---|---|
| | [mmHg] | [mmHg] | [mmHg/s] | (LV + S) | [ml/min] |
| Controls | 29 | 2 | 1629 | 0.26 | 129 |
| Placebo | 69 | 6 | 3397 | 0.49 | 79 |
| Example 1 | 55 | 4 | 2715 | 0.41 | 117 |
| [3-9 mg/ | | | | | |
| kg/d] | | | | | |
| Example 1 | 44 | 3 | 2244 | 0.33 | 117 |
| [10-30 | | | | | |
| mg/kg/d] | | | | | |

TABLE 2

| | RVPmax | RVEDP | dP/dtmax | RV/ | CO |
|---|---|---|---|---|---|
| | [mmHg] | [mmHg] | [mmHg/s] | (LV + S) | [ml/min] |
| Controls | 27 | 2 | 1870 | 0.25 | 124 |
| Placebo | 78 | 5 | 4454 | 0.52 | 99 |
| Example 1 | 57 | 3 | 3300 | 0.38 | 125 |
| [10-30 mg/ | | | | | |
| kg/d] | | | | | |
| Enoxaparin | 85 | 6 | 4644 | 0.56 | 77 |
| [20 mg/kg/d] | | | | | |
| Warfarin | 64 | 4 | 4535 | 0.46 | 100 |
| [0.1-0.2 mg/ | | | | | |
| kg/d] | | | | | |

### d) Hypoxia Model

The experiment is carried out in rodents, e.g. rats or mice. Rats (e.g. Sprague-Dawley; body weight 200-250 g) or mice (e.g. C57/BL6N; body weight 18-20 g) are kept in a controlled hypoxic atmosphere (10% oxygen). Corresponding control rats or mice are kept under normoxic conditions. Chronic hypoxia for at least 14 days leads to the development of a functionally and morphologically detectable pulmonary hypertension in rats and mice (reference: Dumitrascu et al, Circulation 2006; Koulmann et al, Am J Respir Crit. Care Med 2006; Earley et al, Am J Physiol 2002).

Treatment of the animals (by gavage or by adding the test substances in the feed or drinking water or via osmotic minipumps) begins before or at the start of the keeping in a controlled hypoxic atmosphere and extends over a period of at least 14 days.

At the end of the study, the animals undergo haemodynamic investigations (Powerlab Systems, Chart 5 Software, ADinstruments GmbH, Spechbach) under isoflurane anaesthesia (1.6-2% vol/vol, 50% oxygen). The systemic blood pressure is measured in the left carotid artery by means of a Millar microtip catheter (Millar SPR-320 2F for rats and SPR 671 for mice). A polyethylene catheter (rat) or Millar catheter (mouse, Millar SPR 671) is advanced through the right jugular vein into the right ventricle for determining the right ventricular pressure. Following the haemodynamics, the heart is removed and, to determine the right ventricular hypertrophy, the weight ratio of right to left ventricle including septum is determined. In addition, plasma samples are obtained to determine plasma biomarkers and plasma substance levels.

### C. Exemplary Embodiments of Pharmaceutical Compositions

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

Production:

A mixture of compound according to the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

Production:

The compound according to the invention is suspended in the mixture of polyethylene glycol and polysorbate with stirring. The stirring process is continued until the compound according to the invention has completely dissolved.

### I.V. Solution:

The compound according to the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

1. A method for the treatment and/or prophylaxis of pulmonary hypertension comprising administering a therapeutically effective amount of a compound of the formula 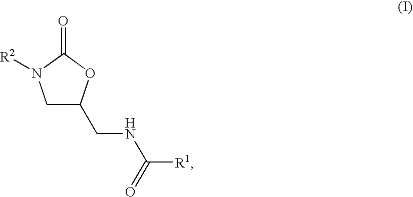
in which
R¹ is 2-thiophene which is substituted in position 5 by a radical selected from the group of chlorine, bromine, methyl and trifluoromethyl,
R² is D-A-,
where
the radical “A” is phenylene,
where
the group “A” may be substituted where appropriate once or twice in the meta position relative to the linkage to the oxazolidinone by a radical selected from the group of fluorine, chlorine, nitro, amino, trifluoromethyl, methyl and cyano,
and
the radical “D” is a saturated 5- or 6-membered heterocycle which is linked via a nitrogen atom to “A” and has in the direct vicinity of the linking nitrogen atom a carbonyl group, and in which one ring carbon member may be replaced by a heteroatom from the series S, N and O,
or one of the salts, solvates and solvates of the salts thereof
to a human or animal patient in need thereof.

2. The method of claim 1, wherein the compound of the formula (I) is 5-chloro-N-({(5S)-2-oxo-3-[4-(3-oxomorpholin-4-yl)phenyl]-1,3-oxazolidin-5-yl}-methyl)thiophene-2-carboxamide of the formula 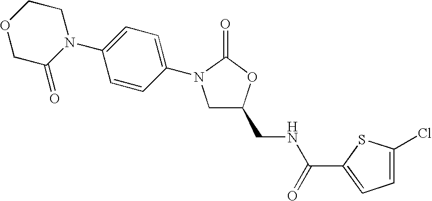
or one of the salts, solvates and solvates of the salts thereof.

3.-6. (canceled)

7. A method for the treatment and/or prophylaxis of pulmonary arterial hypertension comprising administering a therapeutically effective amount of a compound of the formula 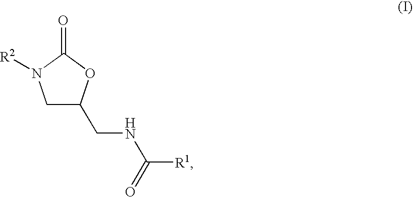
in which
R¹ is 2-thiophene which is substituted in position 5 by a radical selected from the group of chlorine, bromine, methyl and trifluoromethyl,
R² is D-A-,
where
the radical “A” is phenylene,
where
the group “A” may be substituted where appropriate once or twice in the meta position relative to the linkage to the oxazolidinone by a radical selected from the group of fluorine, chlorine, nitro, amino, trifluoromethyl, methyl and cyano,
and
the radical “D” is a saturated 5- or 6-membered heterocycle which is linked via a nitrogen atom to “A” and has in the direct vicinity of the linking nitrogen atom a carbonyl group, and in which one ring carbon member may be replaced by a heteroatom from the series S, N and O,
or one of the salts, solvates and solvates of the salts thereof
to a human or animal patient in need thereof.

8. The method of claim 7, wherein the compound of the formula (I) is 5-chloro-N-({(5S)-2-oxo-3-[4-(3-oxomorpholin-4-yl)phenyl]-1,3-oxazolidin-5-yl}-methyl)thiophene-2-carboxamide of the formula 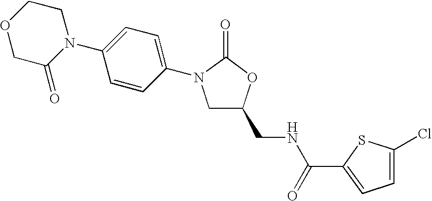
or one of the salts, solvates and solvates of the salts thereof.

9. A method for the treatment and/or prophylaxis of idiopathic pulmonary arterial hypertension, familial pulmonary arterial hypertension (FPAH) and associated pulmonary arterial hypertension (APAH) which is associated with collagenoses, congenital systemic-pulmonary shunts, portal hypertension, HIV infections, intake of particular drugs and medicaments, with other disorders such as thyroid disorders, glycogen storage diseases, Gaucher's disease, hereditary telangiectasia, haemoglobinopathies, myeloproliferative disorders and splenectomy, or with disorders with significant venous/capillary involvement such as pulmonary venoocclusive disease and pulmonary capillary haemangiomatosis, and persistent pulmonary hypertension of newborns comprising administering a therapeutically effective amount of a compound of the formula 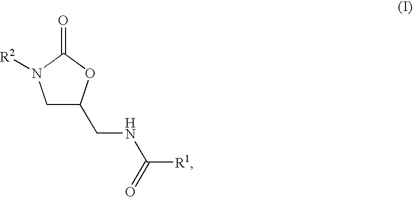
in which
R¹ is 2-thiophene which is substituted in position 5 by a radical selected from the group of chlorine, bromine, methyl and trifluoromethyl,
R² is D-A-,
where
the radical “A” is phenylene,
where
the group “A” may be substituted where appropriate once or twice in the meta position relative to the linkage to the oxazolidinone by a radical selected from the group of fluorine, chlorine, nitro, amino, trifluoromethyl, methyl and cyano,
and
the radical “D” is a saturated 5- or 6-membered heterocycle which is linked via a nitrogen atom to “A” and has in the direct vicinity of the linking nitrogen atom a carbonyl group, and in which one ring carbon member may be replaced by a heteroatom from the series S, N and O,
or one of the salts, solvates and solvates of the salts thereof
to a human or animal patient in need thereof.

10. The method of claim 9, wherein the compound of the formula (I) is 5-chloro-N-({(5S)-2-oxo-3-[4-(3-oxomorpholin-4-yl)phenyl]-1,3-oxazolidin-5-yl}-methyl)thiophene-2-carboxamide of the formula 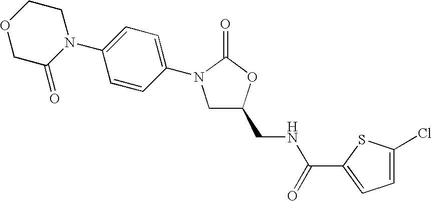
or one of the salts, solvates and solvates of the salts thereof.

11. A method for the treatment and/or prophylaxis of pulmonary hypertension associated with chronic obstructive pulmonary disorders, interstitial lung disease, sleep apnoea syndrome, alveolar hypoventilation, chronic altitude sickness and constitutional abnormalities comprising administering a therapeutically effective amount of a compound of the formula 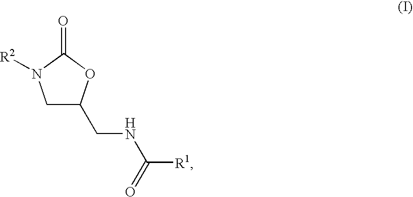
in which
R¹ is 2-thiophene which is substituted in position 5 by a radical selected from the group of chlorine, bromine, methyl and trifluoromethyl,
R² is D-A-,
where
the radical “A” is phenylene,
where
the group “A” may be substituted where appropriate once or twice in the meta position relative to the linkage to the oxazolidinone by a radical selected from the group of fluorine, chlorine, nitro, amino, trifluoromethyl, methyl and cyano,
and
the radical “D” is a saturated 5- or 6-membered heterocycle which is linked via a nitrogen atom to “A” and has in the direct vicinity of the linking nitrogen atom a carbonyl group, and in which one ring carbon member may be replaced by a heteroatom from the series S, N and O,
or one of the salts, solvates and solvates of the salts thereof
to a human or animal patient in need thereof.

12. The method of claim 11, wherein the compound of the formula (I) is 5-chloro-N-({(5S)-2-oxo-3-[4-(3-oxomorpholin-4-yl)phenyl]-1,3-oxazolidin-5-yl}-methyl)thiophene-2-carboxamide of the formula 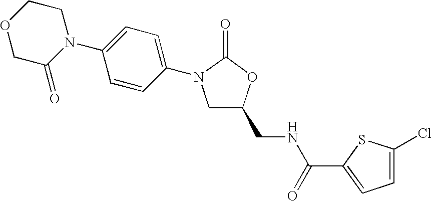
or one of the salts, solvates and solvates of the salts thereof.

13. A method for controlling pulmonary hypertension in humans and animals comprising administering an effective amount of a medicament comprising at least one compound of formula 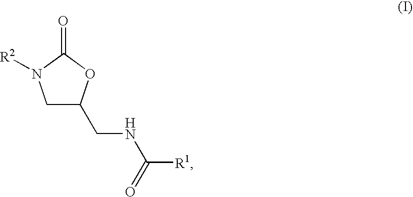
in which
R¹ is 2-thiophene which is substituted in position 5 by a radical selected from the group of chlorine, bromine, methyl and trifluoromethyl,
R² is D-A-,
where
the radical “A” is phenylene,
where
the group “A” may be substituted where appropriate once or twice in the meta position relative to the linkage to the oxazolidinone by a radical selected from the group of fluorine, chlorine, nitro, amino, trifluoromethyl, methyl and cyano,
and
the radical “D” is a saturated 5- or 6-membered heterocycle which is linked via a nitrogen atom to “A” and has in the direct vicinity of the linking nitrogen atom a carbonyl group, and in which one ring carbon member may be replaced by a heteroatom from the series S, N and O,
or one of the salts, solvates and solvates of the salts thereof
in combination with an inert, non-toxic, pharmaceutically suitable excipient to a human or animal patient in need thereof.

14. The method of claim 13, wherein the compound of the formula (I) is 5-chloro-N-({(5S)-2-oxo-3-[4-(3-oxomorpholin-4-yl)phenyl]-1,3-oxazolidin-5-yl}-methyl)thiophene-2-carboxamide of the formula 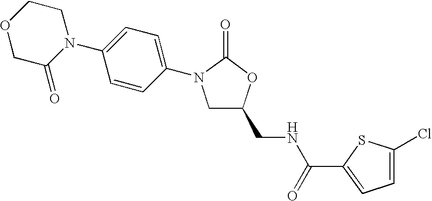
or one of the salts, solvates and solvates of the salts thereof.
